(19) United States
(12) Patent Application Publication
(10) Pub. No.: US 2012/0288909 A1
(43) Pub. Date: Nov. 15, 2012

(54) MICROBIAL PRODUCTION OF PENTANOL FROM GLUCOSE OR GLYCEROL

(75) Inventors: Hsien-Chung TSENG, Cambridge, MA (US); Kristala Lanett Jones Prather, Milton, MA (US)

(73) Assignee: Massachusetts Institute of Technology, Cambridge, MA (US)

(21) Appl. No.: 13/464,441

(22) Filed: May 4, 2012

Related U.S. Application Data
(60) Provisional application No. 61/482,845, filed on May 5, 2011.

(51) Int. Cl. C12N 1/21; C12P 7/04; C12N 5/10

(52) U.S. Cl. 435/157; 435/252.3; 435/419; 435/254.2; 435/254.11; 435/348; 435/325

(57) ABSTRACT

The invention relates to the production of pentanol through recombinant gene expression and metabolic engineering.

### RELATED APPLICATION

This application claims the benefit under 35 U.S.C. §119(e) of U.S. provisional application No. 61/482,845, filed May 5, 2011, the content of which is hereby incorporated by reference in its entirety.

### FIELD OF THE INVENTION

Aspects of the invention relate to the production of pentanol through recombinant gene expression and metabolic engineering.

### BACKGROUND OF THE INVENTION

The increasing demand for biomass-derived fuels has led to a resurgent interest in a number of candidate molecules to complement petroleum-derived transportation fuels. Chief among these, particularly in the U.S., has been ethanol. Ethanol presents a number challenges towards large-scale integration into the fuels supply. Such challenges include an unfavorable carbon balance with the dominant corn-based processes, as well as non-ideal physical-chemical properties, for example, lower energy density compared to existing fuels (e.g., ˜60% that of gasoline). Ethanol is also completely miscible with water, which impacts the distribution infrastructure.

### SUMMARY OF THE INVENTION

Butanol has emerged as the most promising alternative to ethanol because it has a nearly 50% higher energy density than ethanol, representing about 90% of the energy density of gasoline. The primary route for commercial butanol synthesis in the early part of the 20th century was microbial fermentation. The well-known Weizmann or “ABE” process utilizes a species of the Clostridium genus of bacteria, usually Clostridium acetobutylicum, to anaerobically produce a mixture of solvents with a typical ratio of 60% butanol, 30% acetone and 10% ethanol. Research into the process continued in the subsequent years, as the pathway was fully defined and key solvent-producing enzymes identified. Significant advances have also been made with respect to the ability to engineer Clostridium (Ezeji et al. Chem Rec 4, 305-314 (2004); Sillers et al. Metab Eng 10, 321-332 (2008); Tomas et al. Appl Environ Microbiol 69, 4951-4965 (2003)); however, the development of such tools remains substantially behind that of other, more commonly used organisms.

The butanol biosynthetic pathway begins with the condensation of two acetyl-CoA molecules, followed by reduction of the ketone to an (S)-alcohol (FIG. 1). These initial condensation and ketone reduction steps mimic the first two steps for the production of poly(3-hydroxybutyrate-co-3-hydroxyvalerate) (poly(3HB-co-3HV)) co-polymer, one of various polyhydroxyalkanoate biopolymers (PHAs) (FIG. 1), except that the alcoholic ester produced is of the (R)-form. The bacterium Ralstonia eutropha is well-known for its ability to accumulate large amounts of PHAs, including the poly(3HB-co-3HV). A BktB thiolase has been identified within R. eutropha that condenses acetyl-CoA with a longer chain precursor propionyl-CoA to produce five-carbon monomers, and that is distinct from the primary PhaA thiolase that condenses two acetyl-CoA (Slater et al. J Bacteriol 180, 1979-1987 (1998)) (FIG. 1).

Until the present work, the extent to which each of the subsequent enzymes in the butanol pathway will accept five-carbon substrates was unknown. The work presented herein shows that the butanol pathway can be modified for use in producing pentanol.

Certain aspects provided herein relate to a cell(s) that recombinantly expresses one or more genes of the butanol biosynthetic pathway and a gene encoding a thiolase that condenses one acetyl-CoA with one propionyl-CoA to form a ketone.

In some aspects of the invention, the cell(s) recombinantly expresses: (a) a gene encoding a thiolase that condenses one acetyl-CoA with one propionyl-CoA to form a ketone; (b) genes encoding a reductase, a hydratase, and a dehydrogenase that convert the ketone to valeryl-CoA; and (c) a bi-functional aldehyde/alcohol dehydrogenase gene. In some embodiments, the cell(s) recombinantly expresses: (a) a gene that encodes an acetoacetyl-CoA thiolase that condenses one acetyl-CoA with one propionyl-CoA to form 3-ketovaleryl-CoA; (b) genes encoding a 3-hydroxybutyryl-CoA reductase, an enoyl-CoA hydratase, and a butyryl-CoA dehydrogenase; and (c) a gene encoding a bi-functional aldehyde/alcohol dehydrogenase. In other embodiments, the cell(s) recombinantly expresses: (a) a bktB gene; (b) a hbd gene and a crt gene, or a phaB gene and a phaJ1 gene; (c) a bcd-etfAB gene pair; and (d) an adhE gene. In some embodiments, the cell does not express an mdh gene, or an mdh gene is deleted from the cell.

In other aspects of the invention, the cell(s) recombinantly expresses: (a) a gene encoding an acetoacetyl-CoA thiolase that condenses one acetyl-CoA with one propionyl-CoA to form 3-ketovaleryl-CoA; (b) genes encoding a 3-hydroxybutyryl-CoA reductase, an enoyl-CoA hydratase, and a trans-enoyl-CoA reductase; and (c) a gene encoding a bi-functional aldehyde/alcohol dehydrogenase. In some embodiments, the cell(s) recombinantly expresses: (a) a bktB gene; (b) a hbd gene and a crt gene, or a phaB gene and a phaJ1 gene; (c) a Ter gene; and (d) an adhE gene. In some embodiments, the cell does not express an mdh gene, or an mdh gene is deleted from the cell.

In some embodiments, the cell(s) described herein recombinantly express codon-optimized genes. In some embodiments, the adhE gene is codon-optimized.

In some embodiments, the cell(s) recombinantly expresses a thrA^{fr} gene, a thrB gene, a thrC gene, and a ilvA^{fr} gene. In such embodiments, the cell(s) may endogenously or recombinantly express a gene encoding pyruvate dehydrogenase complex (PDHc) or a gene encoding pyruvate-formate lyase (PfIB). In some embodiments, the cell may endogenously or recombinantly express a gene encoding anaerobically active PDHc (or PDHₘ).

In some embodiments, the cell(s) described herein recombinantly expresses a ptb-buk gene pair or a pct gene.

Any cell(s) described in any one of the aspects and/or embodiments presented herein may be a bacterial cell, a fungal cell (including a yeast cell), a plant cell, an insect cell, or an animal cell. In some embodiments, the cell(s) is a bacterial cell. In some embodiments, the bacterial cell(s) is an Escherichia coli cell.

Any gene recombinantly expressed in any cell described in any one of the aspects and/or embodiments presented herein may be expressed from one or more plasmid(s). In some embodiments, one or more of the recombinantly expressed gene(s) is integrated into the genome of the cell.

In some embodiments, the hbd gene, crt gene, bcd-etfAB gene pair, and/or adhE gene(s) described herein is a Clostridium acetobutylicum gene. In some embodiments, the bktB gene and/or phaB gene(s) is a Ralstonia eutropha gene. In such embodiments, the bktB gene and/or phaB gene(s) may be a Ralstonia eutropha H16 gene. In some embodiments, the phaJ1 gene is a Pseudomonas aeruginosa gene. In some embodiments, the Ter gene is a Treponema denticola gene or a Euglena gracilis gene.

Some aspects of the invention are directed to cell culture medium or supernatant collected from culturing one or more cell(s) of any one of aspects and/or embodiments described herein.

Other aspects of the invention are directed to a method, comprising culturing in cell culture medium the cell(s) of any one of the aspects and/or embodiments described herein. In some embodiments, the method comprises feeding valerate to the cell(s). In some embodiments, the method comprises feeding glucose and propionate to the cell(s). In some embodiments, the method comprises feeding glucose and/or glycerol to the cell(s). In the methods of any one of the aspects and/or embodiments presented herein, the headspace to culture volume ratio is about 4 or less.

In some embodiments, the methods of any one of the aspects and/or embodiments described herein may comprise recovering pentanol from the cell(s) or from the culture medium in which the cell(s) is grown.

Various aspects of the invention related to a method, which comprises recombinantly expressing in a cell one or more genes of the butanol biosynthetic pathway and a gene encoding a thiolase that condenses one acetyl-CoA with one propionyl-CoA to form a ketone.

In some aspects, the method comprises recombinantly expressing in the cell(s): (a) a gene encoding a thiolase that condenses one acetyl-CoA with one propionyl-CoA to form a ketone; (b) genes encoding a reductase, a hydratase, and a dehydrogenase that convert the ketone to valeryl-CoA; and (c) a gene encoding a bi-functional aldehyde/alcohol dehydrogenase. In some embodiments, the method comprises recombinantly expressing in the cell(s): (a) a gene encoding an acetoacetyl-CoA thiolase that condenses one acetyl-CoA with one propionyl-CoA to form 3-ketovaleryl-CoA; (b) genes encoding a 3-hydroxybutyryl-CoA reductase, an enoyl-CoA hydratase, and a butyryl-CoA dehydrogenase; and (c) a gene encoding a bi-functional aldehyde/alcohol dehydrogenase. In other embodiments, the methods comprises recombinantly expressing in the cell(s): (a) a bktB gene; (b) a hbd gene and a crt gene, or a phaB gene and a phaJ1 gene; (c) a bcd-etfAB gene pair; and (d) an adhE gene. In some embodiments, the cell does not express an mdh gene, or an mdh gene is deleted from the cell.

In some aspects of the invention, the method comprises recombinantly expressing in a cell(s): (a) a gene encoding an acetoacetyl-CoA thiolase that condenses one acetyl-CoA with one propionyl-CoA to form 3-ketovaleryl-CoA; (b) genes encoding a 3-hydroxybutyryl-CoA reductase, an enoyl-CoA hydratase, and a trans-enoyl-CoA reductase; and (c) a gene encoding a bi-functional aldehyde/alcohol dehydrogenase. In some embodiments, the method comprises recombinantly expressing in the cell(s): (a) a bktB gene; (b) a hbd gene and a crt gene, or a phaB gene and a phaJ1 gene; (c) a Ter gene; and (d) an adhE gene. In some embodiments, the cell does not express an mdh gene, or an mdh gene is deleted from the cell.

In some embodiments, any one of the methods described herein may comprise recombinantly expressing in a cell(s) codon-optimized genes. For example, in some embodiments, the adhE gene is codon-optimized.

In some embodiments, the method comprise recombinantly expressing in a cell(s) a thrA^{fr} gene, a thrB gene, a thrC gene, and a ilvA^{fr} gene. In such embodiments, the cell(s) may endogenously or recombinantly express a gene encoding pyruvate dehydrogenase complex (PDHc) or a gene encoding pyruvate-formate lyase (PfIB). In some embodiments, the cell endogenously or recombinantly expresses a gene encoding anaerobically active PDHc (or PDHₘ).

In some embodiments, the method comprises recombinantly expressing in the cell(s) a ptb-buk gene pair or a pct gene.

Any cell(s) of the methods described in any one of the aspects and/or embodiments presented herein may be a bacterial cell, a fungal cell (including a yeast cell), a plant cell, an insect cell, or an animal cell. In some embodiments, the cell(s) is a bacterial cell. In some embodiments, the bacterial cell(s) is an Escherichia coli cell.

Any gene recombinantly expressed in any cell by the methods described in any one of the aspects and/or embodiments presented herein may be expressed from one or more plasmid(s). In some embodiments, one or more of the recombinantly expressed gene(s) is integrated into the genome of the cell.

In some embodiments, the hbd gene, crt gene, bcd-etfAB gene pair, and/or adhE gene(s) described herein is a Clostridium acetobutylicum gene. In some embodiments, the bktB gene and/or phaB gene(s) is a Ralstonia eutropha gene. In such embodiments, the bktB gene and/or phaB gene(s) may be a Ralstonia eutropha H16 gene. In some embodiments, the phaJ1 gene is a Pseudomonas aeruginosa gene. In some embodiments, the Ter gene is a Treponema denticola gene or a Euglena gracilis gene.

In some embodiments, the methods described in any one of the aspects and embodiments presented herein further comprise culturing in cell culture medium the cell(s) described herein. In some embodiments, the method comprises feeding valerate to the cell(s). In some embodiments, the method comprises feeding glucose and propionate to the cell(s). In some embodiments, the method comprises feeding glucose and/or glycerol to the cell(s). In some embodiments of the methods described herein, the headspace to culture volume ratio is about 4 or less.

In some embodiments, the methods described herein further comprise collecting cell culture medium or supernatant after culturing the cell(s) described herein.

In some embodiments, the methods of any one of the aspects and/or embodiments described herein may comprise recovering pentanol from the cell(s) or from the culture medium in which the cell(s) is grown.

These and other aspects of the invention, as well as various embodiments thereof, will become more apparent in reference to the drawings and detailed description of the invention.

### BRIEF DESCRIPTION OF THE DRAWINGS

FIG. 1 schematically shows the Clostridial butanol biosynthetic pathway (left panel), the poly(3HB-co-3HV) biosynthetic pathway, and the proposed pentanol biosynthetic pathway (right panel). Pentanol synthesis starts from condensation of one acetyl-CoA with one propionyl-CoA, instead of two acetyl-CoA molecules, to establish a five-carbon skeleton. Genes named in the left panel are from C. acetobutylicum while genes named in the right panel are from R. eutropha.

FIG. 2 schematically shows the metabolic pathway and plasmids constructed for direct microbial production of pentanol from glucose or glycerol. Over-expressed genes are shown in larger text in the pathway schematic diagram and are shown in vectors. Four compatible Duet vectors were used to carry all pathway genes.

FIG. 3 schematically shows the trans-2-pentenoate biosynthetic pathway (Top) and titers of products synthesized by recombinant E. coli grown under various conditions (Bottom). All constructs contain ptb-buk and bktB in addition to the genes indicated under each bar. The symbols of C4 and C5 denote crotonate and trans-2-pentenoate, respectively. Ratios of trans-2-pentenoate titers to crotonate titers are shown in the lowest set of numbers (C5/C4) while ratios of titers from phaB-phaJ1 constructs to titers from hbd-crt constructs are shown in the two upper sets of numbers (C4_{(R)}/C4_{(S)} and C5_{(R)}/C5_{(S)}). Cells were grown in TB supplemented with 10 g/L glucose and 20 mM propionate, and incubated at 30° C. for 24 h.

FIG. 4 schematically shows pentanol synthesis from valerate and titers of substrates consumed and products synthesized by recombinant E. coli. Two activators, including Ptb-Buk and Pct, and two feed concentrations of valerate, were compared. The adhEₒₚₜ gene was over-expressed in all constructs.

FIG. 5 schematically shows pentanol synthesis from trans-2-pentenoate and titers of products resulting from the feeding of trans-2-pentenoate. All relevant products coming from trans-2-pentenoate are shown. Genes of ptb-buk, bcd-etfAB and adhEₒₚₜ were over-expressed. Two formate dehydrogenases (encoded by fdh1 with codon-optimization) from Saccharomyces cerevisiae and Candida boidinii were over-expressed to increase availability of NADH. The effect of supplementation with 1 g/L formate was also compared. The calculated total NADH used for product formation was shown within each of bottom three plots.

FIG. 6 schematically shows a comparison of the Clostridial butanol pathway and the newly constructed pentanol pathway. The differences in genes between the Clostridial butanol pathway and the proposed pentanol pathway is shown.

FIG. 7 schematically shows butanol synthesis from glucose via newly constructed pentanol pathways. This figure shows butanol titers, specific titers, and cell densities from cultures of recombinant E. coli containing the pentanol pathways. Cells were grown under various culture conditions with different ratios of headspace to culture volume for 48 h. Two routes, the hbd-crt route (Top) and the phaB-phaJ1 route (Bottom), were compared.

FIG. 8 schematically shows pentanol synthesis from glucose and propionate. Genes of pct and either fdh1 (denoted as Sc) from S. cerevisiae or fdh1 (denoted as Cb) from C. boidinii were over-expressed, in addition to the core pentanol pathway genes. Titers of pentanol and other relevant products are shown in the plot. The symbols of S and R denote the hbd-crt route and the phaB-phaJ1 route, respectively. The products shown are, from left in each set of bars: acetate (first bar in each set), butyrate (second bar in each set), butanol (third bar in each set), propanol (fourth bar in each set), valerate (fifth bar in each set), and pentanol (sixth bar in each set).

FIG. 9 schematically shows pentanol synthesis solely from glucose or glycerol. A pathway allowing for endogenous supply of propionyl-CoA was introduced along with over-expression of either fdh1 (denoted as Sc) from S. cerevisiae or fdh1 (denoted as Cb) from C. boidinii, in addition to the core pentanol pathway genes. The symbols of S and R denote the hbd-crt route and the phaB-phaJ1 route, respectively. The relative redox values of relevant products are shown on the left panel and the calculated total NADH used for product formation was shown above each data set on the right panel. The products shown in the right panel are, from left in each set of bars: propionate (first bar in each set), propanol (second bar in each set), butyrate (third bar in each set), butanol (fourth bar in each set), and valerate (fifth bar in each set).

FIG. 10 schematically shows correlations between dissolved oxygen and various variables (cofactor ratios, ATP, and observed product ratios).

FIG. 11 schematically shows the metabolic pathway and sub-pathways (“modules”) for direct microbial production of pentanol from glucose or glycerol.

FIG. 12A shows pentanol and valerate synthesis from glucose and trans-2-pentenoate. FIG. 12B shows pentanol and valerate synthesis from glucose and propionate using modules 2R and 3 (top) or modules 2S and 3 (bottom). FIG. 12C shows production of various alcohols from glucose or glycerol. FIG. 12D shows production of various alcohols from glycerol in mutant cells (without the mdh gene or the adhE gene).

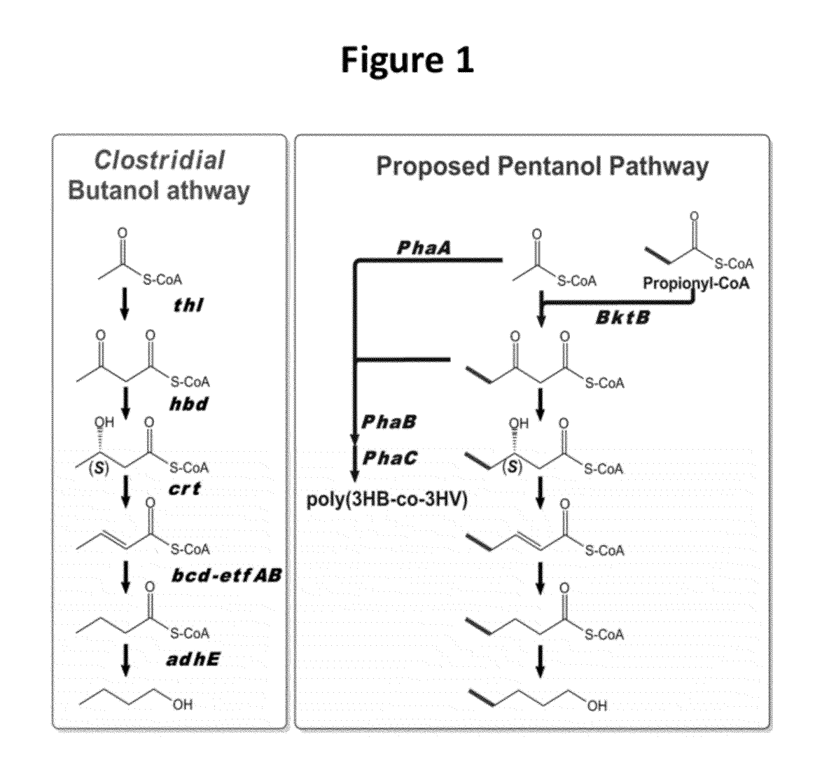

### DETAILED DESCRIPTION OF SOME EMBODIMENTS OF THE INVENTION

Described herein is the surprising discovery that pentanol can be produced through a biological process involving metabolic engineering by recombinant gene expression in cells. Methods and compositions of the invention relate to the production of pentanol in a cell that recombinantly expresses one or more genes including bktB, thl, phaA, hbd, phaB, crt, phaJ1, bcd-etfAB, Ter, acd, fdh1, ilvA, thrA, and thrB. BktB, for example, functions to form both four- and five-carbon molecules, but its activity is approximately three-fold higher for the latter. Thus, in the absence of a primary thiolase, the predominant products are of the five-carbon variety. Herein, BktB thiolase, which preferentially condenses one acetyl-CoA and one propionyl-CoA, was incorporated in place of C. acetobutylicum Thl thiolase into the basic butanol pathway for microbial synthesis of pentanol, a biofuel useful for many applications.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the invention relate to recombinant expression of one or more genes encoding for one or more enzymes in a pentanol biosynthetic pathway. Enzymes associated with this pathway include thiolases (encoded by bktB, thl, or phaA), reductases (encoded by hbd, phaB, or Ter), hydratases (encoded by crt or phaJ1), and dehydrogenases (encoded by bcd-etfAB, acd, adhE, or fdh1). Some aspects relate to recombinant expression of thiolase (encoded by bktB), (S)-3-hydroxybutyryl-CoA reductase (encoded by hbd), (S)-Enoyl-CoA hydratase (encoded by crt), and bi-functional aldehyde/alcohol dehydrogenase (encoded by adhE). Other aspects relate to recombinant expression of thiolase (encoded by bktB), (R)-3-hydroxybutyryl-CoA reductase (encoded by phaB), (R)-Enoyl-CoA hydratase (encoded by phaJ1), and bi-functional aldehyde/alcohol dehydrogenase (encoded by adhE). In some embodiments, a cell associated with the invention also recombinantly expresses a gene encoding for butyryl-CoA dehydrogenase (e.g., bcd-etfAB). In other embodiments, a cell associated with the invention also recombinantly expresses a gene encoding for trans-2-enoyl-CoA reductase (e.g., Ter).

According to aspects of the invention, cell(s) that recombinantly express one or more genes described herein, and the use of such cells in producing pentanol are provided. It should be appreciated that the genes described herein can be obtained from a variety of sources. In some embodiments, the phaA, phaB, and bktB genes are obtained from a strain of Ralstonia eutropha, such as Ralstonia eutropha H16, the hbd, thl, crt, bcd-etfAB, and adhE genes are obtained from a strain of C. acetobutylicum, such as C. acetobutylicum 824, the phaJ1 gene is obtained from a strain of Pseudomonas aeruginosa, the Ter gene is obtained from a strain of Treponema denticola (see Tucci et al. FEBS Letters 581, 1561-1566 (2007)) or Euglena gracilis (Hoffmeister et al. J. Biol. Chem. 280(6), 4329-4338 (2005)), the acd gene is obtained from a strain of Pseudomonas putida, and the fdh1 gene is obtained from either Candida boidinii or Saccharomyces cerevisiae. In some embodiments, the sequence of the phaA gene is represented by GenBank accession no. P14611 (Peoples and Sinskey, 1989), the sequence of the phaB gene is represented by GenBank accession no. P14697 (Peoples and Sinskey, 1989).

As one of ordinary skill in the art would be aware, homologous genes for these enzymes could be obtained from other species and could be identified by homology searches, for example through a protein BLAST search, available at the National Center for Biotechnology Information (NCBI) internet site (www.ncbi.nlm.nih.gov). Genes associated with the invention can be PCR amplified from DNA from any source of DNA which contains the given gene. In certain embodiments, genes derived from C. acetobutylicum ATCC 824 (hbd, crt, bcd, etfAB, adhE, and ptb-buk), R. eutropha H16 (bktB and phaB), P. aeruginosa (phaJ1), M. elsdenii (pct), C. glutamicum ATCC 13032 (ilvA), E. coli ATCC 21277 (thrA^{G1297A}BC operon), E. gracilis (Ter), and T. denticola (Ter) are obtained by polymerase chain reaction (PCR) using genomic DNA (gDNA) templates. In some embodiments, genes associated with the invention are synthetic. Any means of obtaining a gene encoding the enzymes associated with the invention are compatible with the instant invention.

The invention encompasses any type of cell that recombinantly expresses genes associated with the invention, including prokaryotic and eukaryotic cells. In some embodiments the cell is a bacterial cell. In some embodiments the bacterial cell is an Escherichia coli (E. coli) cell. In other embodiments the cell is a fungal cell such as yeast cells, e.g., S. cerevisiae. In other embodiments the cell is a mammalian cell or a plant cell. It should be appreciated that some cells compatible with the invention may express an endogenous copy of one or more of the genes associated with the invention as well as a recombinant copy. In some embodiments if a cell has an endogenous copy of one or more of the genes associated with the invention then the methods will not necessarily require adding a recombinant copy of the gene(s) that are endogenously expressed. In some embodiments the cell may endogenously express one or more enzymes from the pathways described herein and may recombinantly express one or more other enzymes from the pathways described herein for efficient production of pentanol.

In some embodiments, one or more of the genes associated with the invention is expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA although RNA vectors are also available. Vectors include, but are not limited to: plasmids, fosmids, phagemids, virus genomes and artificial chromosomes. In particular embodiments, pETDuet-1, pCDFDuet-1, pACYCDuet-1, and pCOLADuet-1 (Novagen, Darmstadt, Germany) are used to provide individual expression of each gene under a T7lac promoter and a ribosome binding site (RBS).

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the enzymes of the claimed invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell. Heterologous expression of genes associated with the invention, for production of pentanol is demonstrated in the Examples section using E. coli. In certain embodiments E. coli strain DH10B is used, while in other embodiments E. coli ElectroTen-Blue is used. In certain other embodiments, plasmids are co-transformed into E. coli BL21Star(DE3) or Pal(DE3) to create production strains. The novel method for producing pentanol can also be expressed in other bacterial cells, archaeal cells, fungi (including yeast cells), mammalian cells, plant cells, etc.

A nucleic acid molecule that encodes the enzyme of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

In some embodiments one or more genes associated with the invention is expressed recombinantly in a bacterial cell. Bacterial cells according to the invention can be cultured in media of any type (rich or minimal) and any composition. As would be understood by one of ordinary skill in the art, in some embodiments, routine optimization would allow for use of a variety of types of media. The selected medium can be supplemented with various additional components. Some non-limiting examples of supplemental components include glucose, glycerol, antibiotics, IPTG for gene induction, and ATCC Trace Mineral Supplement. Similarly, other aspects of the medium, and growth conditions of the cells of the invention may be optimized through routine experimentation. For example, pH and temperature are non-limiting examples of factors which can be optimized. In some embodiments, factors such as choice of media, media supplements, and temperature can influence production levels of pentanol. In some embodiments the concentration and amount of a supplemental component may be optimized. In some embodiments, how often the media is supplemented with one or more supplemental components, and the amount of time that the media is cultured before harvesting pentanol is optimized.

In some embodiments the methods associated with the invention, for the production of pentanol, can be modified to use glucose or glycerol, or a combination of glucose and glycerol. In other embodiments, other feedstock/carbon sources are used, for example, valerate or propionate. In certain embodiments, the media is supplemented with both glucose and propionate.

According to aspects described herein, high titers of pentanol are produced through the recombinant expression of genes associated with the invention, in a cell. As used herein “high titer” refers to a titer in the milligrams per liter (mg L⁻¹) scale. The titer produced for a given product will be influenced by multiple factors including choice of media. In some embodiments the titer for production of pentanol is at least 25 mg L⁻¹ in minimal media. For example the titer may be 25, 50, 75, 100, 125, 150, 175, 200, 225, 250, 275, 300 or more than 300 mg L⁻¹ including any intermediate values. In some embodiments the titer for production of pentanol is at least 200 mg L⁻¹ in rich media. For example the titer may be 200, 225, 250, 275, 300, 325, 350, 375, or more than 375 mg L⁻¹ including any intermediate values. In some embodiments the titer for production of pentanol is at least 1 mg L⁻¹ in minimal media. For example the titer may be 1, 2, 3, 4, 5, 6, 7, 8, 9, 10, 15, 20, 25, 30, 35, 40, 45, 50, or more than 50 mg L⁻¹ including any intermediate values. In some embodiments the titer for production of pentanol is at least 1 mg L⁻¹ in rich media. For example the titer may be 1, 2, 3, 4, 5, 6, 7, 8, 9, 10, 15, 20, 25, 30, 35, 40, 45, 50, or more than 50 mg L⁻¹ including any intermediate values.

In certain aspects, liquid cultures used to grow cells associated with the embodiments described herein are housed in any of the culture vessels known and used in the art. In some embodiments large scale production in an aerated reaction vessel such as a stirred tank reactor is used to produce large quantities of pentanol.

Aspects of the invention include strategies to optimize production of pentanol from a cell. Optimized production of pentanol refers to producing a higher amount of a pentanol following pursuit of an optimization strategy than would be achieved in the absence of such a strategy. In some embodiments, optimization includes increasing expression levels of one or more genes described herein through selection of appropriate promoters and ribosome binding sites. In some embodiments this includes the selection of high-copy number plasmids, or low or medium-copy number plasmids. In some embodiments the plasmid is a medium-copy number plasmid such as pETDuet. Other plasmids that can be used in the cells and methods described herein include pCDFDuet-1, pACYCDuet-1, and pCOLADuet-1. The step of transcription termination may also be targeted in some embodiments for regulation of gene expression, through the introduction or elimination of structures such as stem-loops.

In some embodiments, a cell that has been optimized for production of pentanol is used. In some embodiments, screening for mutations that lead to enhanced production of pentanol is conducted through a random mutagenesis screen, or through screening of known mutations. In other embodiments shotgun cloning of genomic fragments is used to identify genomic regions that lead to an increase in production of pentanol, through screening cells or organisms that have these fragments for increased production of pentanol. In some cases one or more mutations are combined in the same cell or organism.

Optimization of protein expression may also require in some embodiments that a gene encoding an enzyme be modified before being introduced into a cell such as through codon optimization for expression in a bacterial cell. Codon usages for a variety of organisms can be accessed in the Codon Usage Database (website: kazusa.or.jp/codon/). In certain embodiments, codon-optimized genes include adhE from C. acetobutylicum ATCC 824, fdh1 from S. cerevisiae, and fdh1 from C. boidinii.

In some embodiments protein engineering can be used to optimize expression or activity of one or more enzymes associated with the invention. In certain embodiments a protein engineering approach could include determining the 3D structure of an enzyme or constructing a 3D homology model for the enzyme based on the structure of a related protein. Based on 3D models, mutations in an enzyme can be constructed and incorporated into a cell or organism, which could then be screened for an increased production of pentanol. In some embodiments production of pentanol in a cell is increased through manipulation of enzymes that act in the same pathway as the enzymes associated with the pathways described herein. For example, in some embodiments it may be advantageous to increase expression of an enzyme or other factor that acts upstream of a target enzyme such as an enzyme associated with any one or more of the pathways described herein. In some embodiments, this is achieved by over-expressing the upstream factor using any standard method.

In certain embodiments, provided herein is a cell or cells that produce pentanol via one or more biosynthetic pathways. In some embodiments, the cell(s) recombinantly express one or more genes of the butanol biosynthetic pathway and a gene encoding a thiolase that condenses one acetyl-CoA with one propionyl-CoA to form a ketone. In particular aspects, the thiolase gene is an acetoacetyl-CoA thiolase gene, for example, a bktB gene. In particular embodiments, the thiolase gene may be a thl gene. In other embodiments, the cell(s) recombinantly express (a) a gene encoding a thiolase that condenses one acetyl-CoA with one propionyl-CoA to form a ketone; (b) a reductase, a hydratase, and a dehydrogenase that convert the ketone to valeryl-CoA; and (c) a bi-functional aldehyde/alcohol dehydrogenase.

In particular embodiments, the cell(s) recombinantly express (a) an acetoacetyl-CoA thiolase gene encoding a thiolase that condenses one acetyl-CoA with one propionyl-CoA to form 3-ketovaleryl-CoA; (b) a 3-hydroxybutyryl-CoA reductase, an enoyl-CoA hydratase, and a butyryl-CoA dehydrogenase; and (c) a bi-functional aldehyde/alcohol dehydrogenase. In other embodiments, the cell(s) recombinantly express (a) an acetoacetyl-CoA thiolase gene encoding a thiolase that condenses one acetyl-CoA with one propionyl-CoA to form 3-ketovaleryl-CoA; (b) a 3-hydroxybutyryl-CoA reductase, an enoyl-CoA hydratase, and a trans-enoyl-CoA reductase; and (c) a bi-functional aldehyde/alcohol dehydrogenase.

In some embodiments, the cell(s) recombinantly express (a) a bktB gene; (b) a hbd gene and a crt gene, or a phaB gene and a phaJ1 gene; (c) a bcd-etfAB gene pair or a Ter gene; and (d) an adhE gene. In certain embodiments, the adhE gene is codon-optimized.

In certain embodiments, the cell(s) recombinantly express (a) a bktB gene; (b) a hbd gene and a crt gene, or a phaB gene and a phaJ1 gene; (c) a bcd-etfAB gene pair or a Ter gene; (d) an adhE gene; and (e) a thrA^{fr} gene, a thrB gene, a thrC gene, and a ilvA^{fr} gene. In yet other embodiments, the cell(s) recombinantly express (a) a bktB gene; (b) a hbd gene and a crt gene, or a phaB gene and a phaJ1 gene; (c) a bcd-etfAB gene pair or a Ter gene; (d) an adhE gene; (e) thrA^{fr} gene, a thrB gene, a thrC gene, and a ilvA^{fr} gene; and (f) a gene encoding pyruvate dehydrogenase complex (PDHc) or a gene encoding pyruvate-formate lyase (PfIB).

In still other embodiments, the cell(s) recombinantly express (a) a bktB gene; (b) a hbd gene and a crt gene, or a phaB gene and a phaJ1 gene; (c) a bcd-etfAB gene pair or a Ter gene; and (d) an adhE gene; (e) a thrA^{fr} gene, a thrB gene, a thrC gene, and a ilvA^{fr} gene; (f) a gene encoding pyruvate dehydrogenase complex (PDHc) or a gene encoding pyruvate-formate lyase (PfIB); and (g) a ptb-buk gene pair or a pct gene. In certain embodiments, these cell(s) are cultured in media supplemented with glycerol or glucose or both.

In some embodiments, the cell(s) recombinantly express a ptb-buk gene pair; a bktB gene; and a hbd gene and a crt gene, or a phaB gene and a phaJ1 gene. In certain embodiments, these cell(s) are cultured in media supplemented with glucose and propionate.

In certain embodiments, the cell(s) recombinantly express activation enzymes buk, pct, ptb, and codon-optimized adhE. In some embodiments, these cell(s) are cultured in media supplemented with valerate.

In other embodiments, the cell(s) recombinantly express a ptb-buk gene pair, codon-optimized adhE, and a bcd-etfAB gene pair. In particular embodiments, the cell(s) also recombinantly express fdh1.

### EXAMPLES

A bypass strategy was used to examine the ability of the enzymes employed in the proposed pentanol biosynthetic pathway to accept five-carbon substrates. Particularly, certain coenzyme A (CoA) derivatives synthesized via reduction reactions along the pentanol pathway were targeted to be converted to their respective free acid forms, allowing for their extracellular detection. Alternatively, certain carboxylic acids were fed to serve as precursors of targeted CoA intermediates, both which can be achieved through the use of CoA-addition/removal tools, including broad-substrate-range enzymes of Ptb-Buk (from C. acetobutylicum) and TesB (from E. coli).

### Example 1

### Construction of Pentanol Biosynthetic Pathway

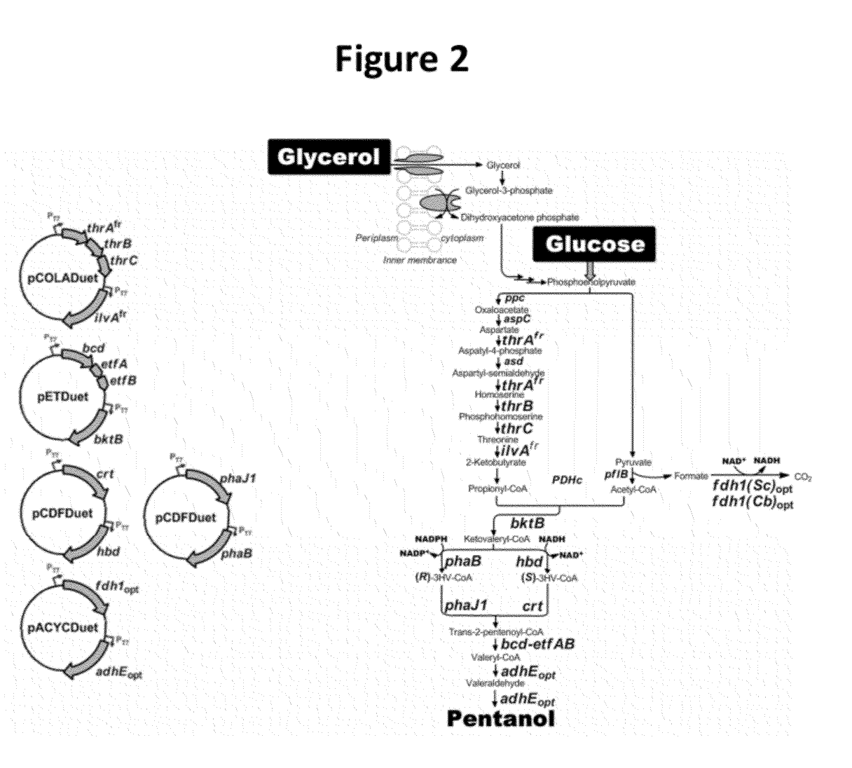

A synthetic pathway for production of pentanol has been constructed. This pathway combines elements of the Clostridial butanol biosynthesis pathway with pathways of the poly(3HB-co-3HB) biosynthesis of R. eutropha and threonine biosynthesis of E. coli (FIG. 2). Pentanol biosynthesis begins with condensation of one acetyl-CoA and one propionyl-CoA to form 3-ketovaleryl-CoA. This reaction is catalyzed by an acetoacetyl-CoA thiolase from R. eutropha H16, which is encoded by bktB. The genes encoding for enzyme activities for the step-wise conversion of 3-ketovaleryl-CoA to valeryl-CoA are clustered together in a polycistronic operon, consisted of genes crt, bcd, etfAB, and hbd from C. acetobutylicum, encoding for crotonase, butyryl-CoA dehydrogenase, electron transfer proteins, and 3-hydroxybutyryl-CoA dehydrogenase, respectively. A bi-functional aldehyde/alcohol dehydrogenase, encoded by adhE from C. acetobutylicum, catalyzes the final steps of pentanol synthesis from valeryl-CoA. Alternatively, both hbd and crt genes can be replaced with phaB from R. eutropha H16 and phaJ1 from Pseudomonas aeruginosa, respectively, to convert ketovaleryl-CoA to trans-2-pentenoyl-CoA. See Table 1 for a complete list of alternative genes for pentanol synthesis. While acetyl-CoA is an obligate central intermediate occurring in any organism and under any physiological condition, this is not the case for propionyl-CoA. Given that synthesis of 3-ketovaleryl-CoA requires propionyl-CoA biosynthesis, a pathway allowing for endogenous propionyl-CoA synthesis from glucose or glycerol was also introduced. Specifically, up-regulation of threonine biosynthesis by over-expressing an E. coli thrA^{fr}BC operon along with over-expression of avg (encoding threonine deaminase) was performed to enhance synthesis of 2-ketobutyrate, a common keto-acid intermediate for isoleucine biosynthesis. 2-ketobutyrate can further be converted to propionyl-CoA by an endogenous pyruvate dehydrogenase complex (PDHc) or pyruvate-formate lyase (PfIB).

TABLE 1
List of alternative genes for pentanol synthesis

| Enzymes | Genes | Source Organisms |
|---|---|---|
| Thiolase (acetyl-CoA | bktB | Ralstonia eutropha |
| acetyltransferase) | thl | Clostridium acetobutylicum |
| | phaA | Ralstonia eutropha |
| (S)-3-Hydroxybutyryl-CoA | hbd | Clostridium acetobutylicum |
| reductase | | |
| (R)-3-Hydroxybutyryl-CoA | phaB | Ralstonia eutropha |
| reductase | | |
| (S)-Enoyl-CoA hydratase | crt | Clostridium acetobutylicum |
| (R)-Enoyl-CoA hydratase | phaJ1 | Pseudomonas aeruginosa |
| Butyryl-CoA dehydrogenase | bcd-etfAB | Clostridium acetobutylicum |
| Trans-enoyl-CoA reductase | Ter | Treponema denticola or |
| | | Euglena gracilis |
| Acyl-CoA dehydrogenase | acd | Pseudomonas putida |
| Bi-functional aldehyde/alcohol | adhE | Clostridium acetobutylicum |
| dehydrogenase | | |
| Formate dehydrogenase | fdh1 | Candida boidinii |
| | fdh1 | Saccharomyces cerevisiae |

### Example 2

### Trans-2-pentenoate Synthesis from Glucose and Propionate

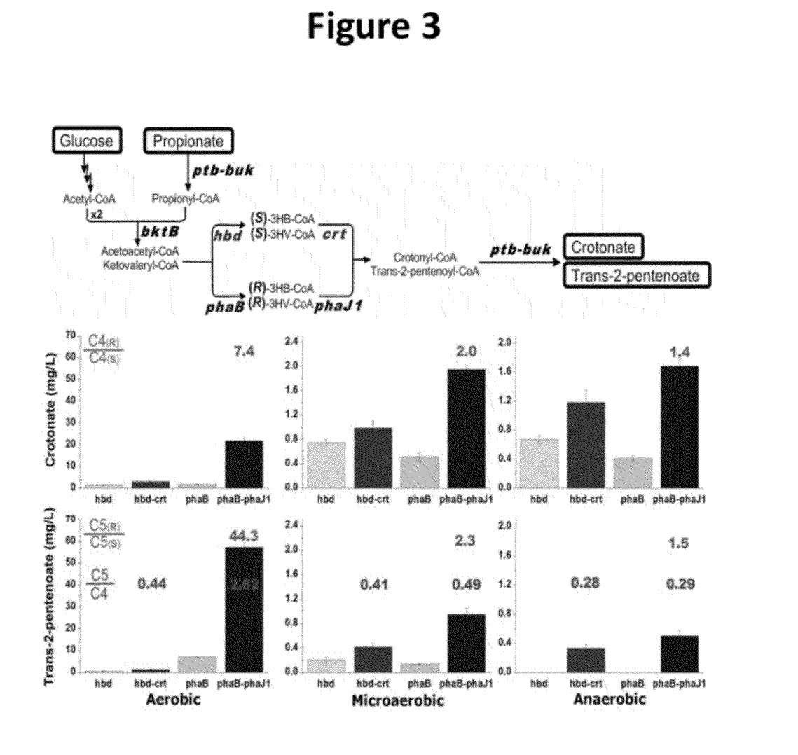

To evaluate the ability of upstream pentanol pathway enzymes to accept five-carbon substrates, the pentanol pathway was shortcut towards production of trans-2-pentenoate along with over-expression of a CoA activator Ptb-Buk. Here, two distinct metabolic routes, including one through (S)-3HV-CoA with an hbd-crt gene pair and the other through (R)-3HV-CoA with a phaB-phaJ1 gene pair, were examined (FIG. 3).

Strains expressing phaJ1 along with phaB were able to produce much more crotonate and trans-2-pentenoate than the no-phaJ1 control under aerobic, microaerobic, and anaerobic conditions. Strains expressing crt along with hbd also produced both crotonate and trans-2-pentenoate, but the difference in titers between crt-expressing and crt-lacking strains was small. In general, there existed some background crotonase activity (both S- and R-specific) that resulted in background production of crotonate and trans-2-pentenoate as observed in the no-crt and no-phaJ1 controls. Altogether, the results show that all enzymes examined here, particularly Hbd, Crt, PhaB and PhaJ1, accepted five-carbon substrate analogues in addition to the natural four-carbon substrates.

To compare the two routes employed for the trans-2-pentenoate synthesis, two ratios, including one ratio of product titers from phaB-phaJ1 constructs to those from hbd-crt and the other ratio of tran-2-pentenoate titers to crotonate titers (FIG. 3) were calculated. For the ratios of product titers from phaB-phaJ1 constructs to those from hbd-crt, a value larger than one suggests that the R-pathway outperforms the S-pathway, which was the case under aerobic conditions. On the other hand, the ratio was close to one under anaerobic conditions, suggesting that the difference between the R- and S-pathway became smaller. For the ratios of tran-2-penenote titers to crotonate titers, the values also dropped when culture condition changes from aerobic to anaerobic. The difference in the two ratios between the R- and S-pathway implicates a correlation between oxygen availability and R- and S-pathway activities.

### Example 3

### Pentanol Synthesis from Valerate

After validating the top pentanol pathway through synthesis of trans-2-pentenoate, the next experiment was designed to evaluate whether the bottom pentanol pathway enzymes, including the butyryl-CoA dehydrogenase (encoded by bcd-etfAB) and the bi-functional alcohol/aldehyde dehydrogenase (encoded by adhE), can accept non-natural five-carbon substrates.

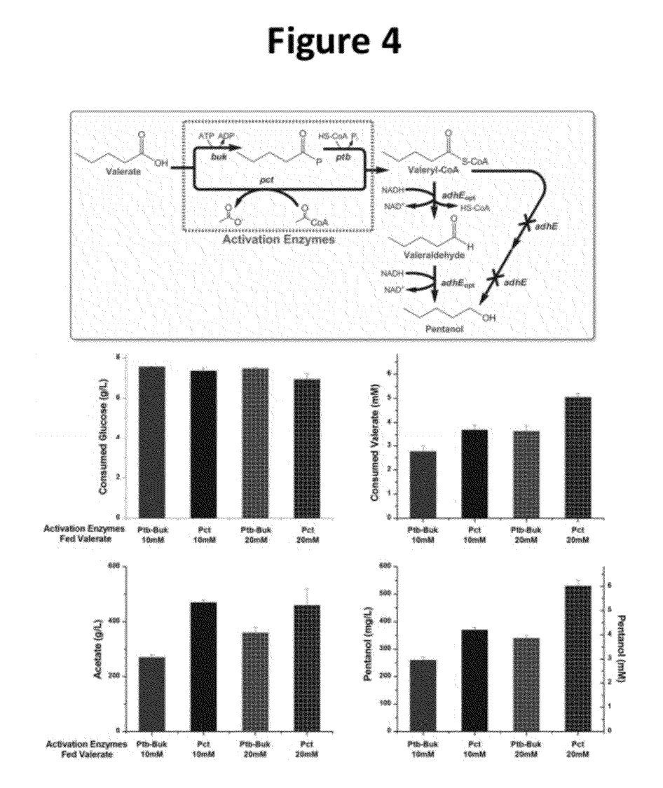

Valerate was initially supplemented to the culture in addition to glucose to test whether the AdhE enzyme could convert valeryl-CoA to pentanol (FIG. 4). Here, two versions of AdhE coding genes, including one original adhE from C. acetobutylicum and one codon-optimized adhE (denoted as adhEₒₚₜ), were explored. Cells containing adhEₒₚₜ along with ptb-buk or pct were able to produce pentanol from valerate, though this was not the case for cells containing adhE. Such a result is consistent with observations on a protein gel showing that expression of adhE was significantly improved by codon-optimization as a strong protein band appeared on the protein gel from the soluble fraction of cell lysate.

In addition, two activators were compared for valerate activation, and stains utilizing Pct as CoA-activation were found to consume more valerate and produce more pentanol compared to stains utilizing Ptb-Buk as CoA-activation. Pentanol titers were further boosted when the valerate substrate concentration was increased from 10 mM to 20 mM (FIG. 4). Overall, the results presented here suggest that the AdhE enzyme, expressed from the codon-optimized adhEₒₚₜ, was able to catalyze the reaction of valeryl-CoA to pentanol.

### Example 4

### Pentanol Synthesis from trans-2-pentenoate

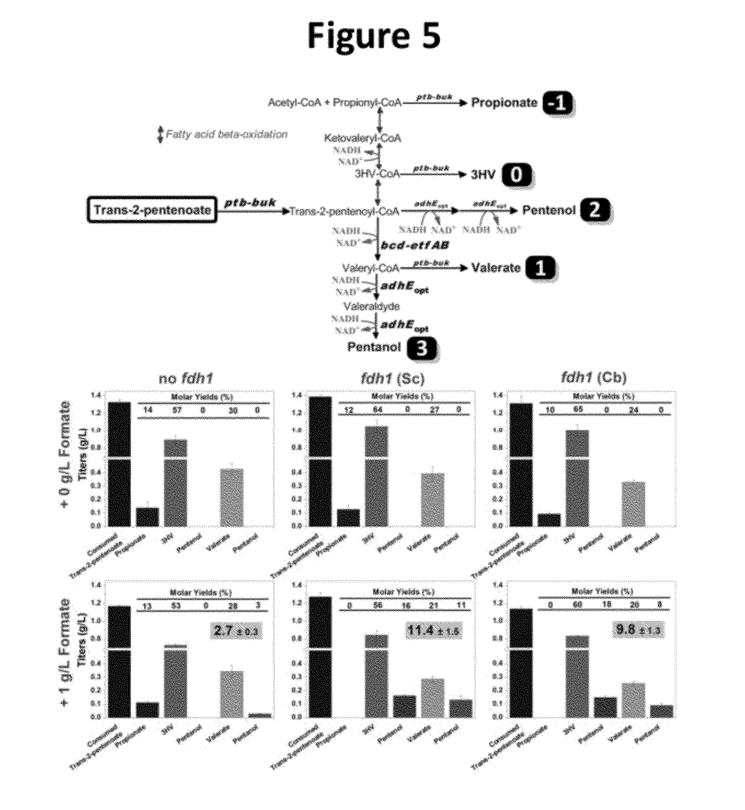

Next, one additional enzymatic step upstream to the AdhE reaction was examined through feeding of trans-2-pentenoate (FIG. 5). Trans-2-pentenoate was first activated to trans-2-pentenoyl-CoA by the activator Ptb-Buk, followed by sequential reduction to pentanol, catalyzed by Bcd and AdhE. Due to substrate promiscuity of those introduced enzymes together with endogenous fatty acid beta-oxidation activities, production of several other metabolites such as propionate, 3HV, pentenol, and valerate, was also expected. Cells containing the bottom pentanol pathway produced valerate with a molar yield of 30% on consumed trans-2-pentenoate, but resulted in undetectable pentanol production (top left plot in FIG. 5). The failure in pentanol production is most likely due to an intensive cofactor requirement of two moles of NADH for the reaction from valeryl-CoA to pentanol as opposed to zero for the reaction from valeryl-CoA to valerate while generating one mole of ATP. Energetically speaking, production of valerate would be more preferable for E. coli as opposed to production of pentanol. If that is the case, an increased NADH availability would be expected to allow for pentanol synthesis. One way to increase NADH availability is to utilize an NAD⁺-dependent formate dyhydrogenase from yeast.

The native E. coli formate dehydrogenase converts formate to CO₂ and H₂ without generation of any NADH while the NAD⁺-dependent formate dehydrogenase from several yeast strains could generate one mole of NADH with conversion of one mole of formate (Berrios-Rivera et al., Metab Eng 4, 217-229 (2002)). It has been demonstrated that over-expression of the NAD⁺-dependent FDH1 from Candida boidinii increased NADH availability by extracting reducing power from formate and, consequently, enhanced ethanol production (Berrios-Rivera et al., 2002).

Herein, two codon-optimized fdh1 genes from Saccharomyces cerevisiae and C. boidinii were employed. The effect of over-expression of the two fdh1 genes was then investigated with the bottom pentanol pathway (schematic in FIG. 5). Because the E. coli host strain used here was BL21Star(DE3), which normally produces little amount of formate under anaerobic conditions, supplementation of formate was required. There was no effect of over-expression of either fdh1 gene when formate was not supplemented (top three plots in FIG. 5). By comparison, in the formate supplemented cultures, the over-expression of fdh1 resulted in synthesis of more reduced products, including pentenol (a mono-unsaturated five-carbon alcohol) and pentanol (bottom three plots in FIG. 5).

To quantify the effect of over-expression of fdh1, one number was assigned to each product based on its relative redox state, for example, 2 for pentenol as its synthesis from trans-2-pentenoyl-CoA requires 2 moles of NADH and −1 for propionate as its synthesis from trans-2-pentenoyl-CoA generates 1 mole of NADH. Next, the total NADH used for product formation was calculated, which is the summation of products of multiplying the relative redox value by the product titer. The over-expression of fdh1 with supplementation of formate increased the NADH availability within the cells as the total NADH used for product formation increased from 2.7 in the no-fdh1 control to 11.4 and 9.8 in cells expressing S. cerevisiae fdh1 and C. boidinii fdh1, respectively.

One control experiment with a strain without bcd-etfAB was conducted to confirm that the Bcd activity came directly from the over-expressed bcd-etfAB operon but not from a background activity of E. coli, as neither pentanol nor valerate was produced in such strain. This and the other observation of production of valerate and pentanol in the bcd-etfAB containing strain suggest that Bcd employed in the bottom pentanol pathway can transform trans-2-pentenoyl-CoA, a five-carbon substrate, to valeryl-CoA.

### Example 5

### Validation of the Pentanol Pathway by Butanol Synthesis

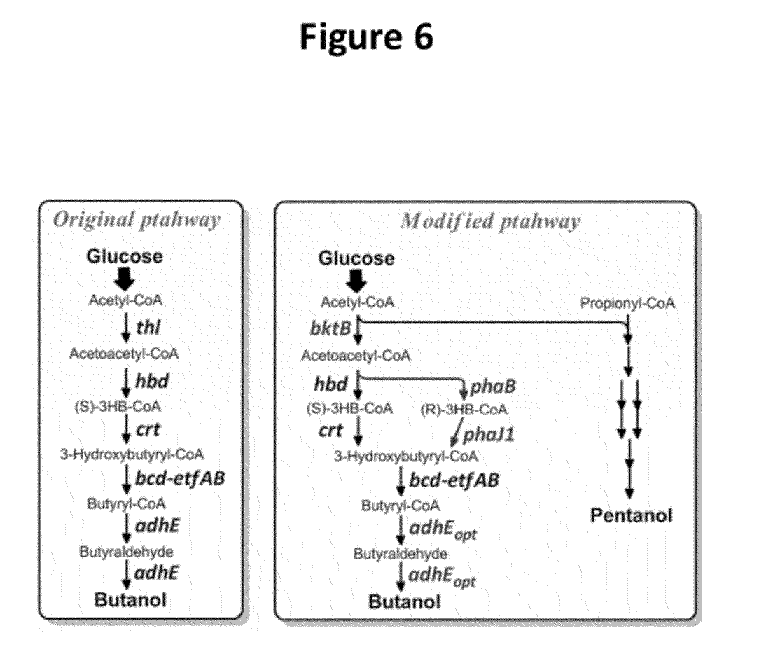

After verifying each of the top and bottom pentanol pathways, they were assembled together. Compared to the original Clostridial butanol pathway (left diagram in FIG. 6), three key changes occurred in our newly constructed pentanol pathway (right diagram in FIG. 6). First, a bktB thiolase with broader substrate specificity was used to do the initial condensation reaction as opposed to thl thiolase. Second, an alternative route through phaB and phaJ1 was constructed in addition to that through hbd and crt. Third, a codon-optimized adhEₒₚₜ gene was used, in place of the original adhE gene, to promote its functional expression in E. coli. To validate the newly constructed pentanol biosynthetic pathways, butanol synthesis was initially examined.

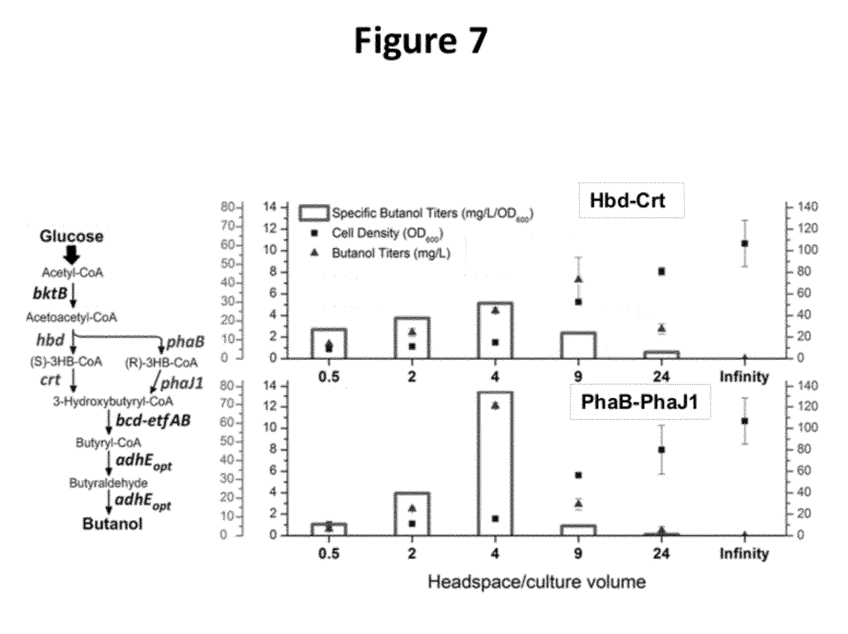

Cells were cultivated in conditions with various ratios of headspace to culture volume for 48 h. The reason of profiling the headspace to culture volume ratios is because of our previous observation on a correlation between oxygen availability and R- and S-pathway activity as seen in the experiment of trans-2-pentenoate production from glucose and propionate (FIG. 3). In general, strains containing either R- or S-pentanol biosynthetic pathway produced butanol, achieving the highest butanol specific titers at a headspace to culture volume ratio of 4 (FIG. 7).

### Example 6

### Pentanol Synthesis from Glucose and Propionate

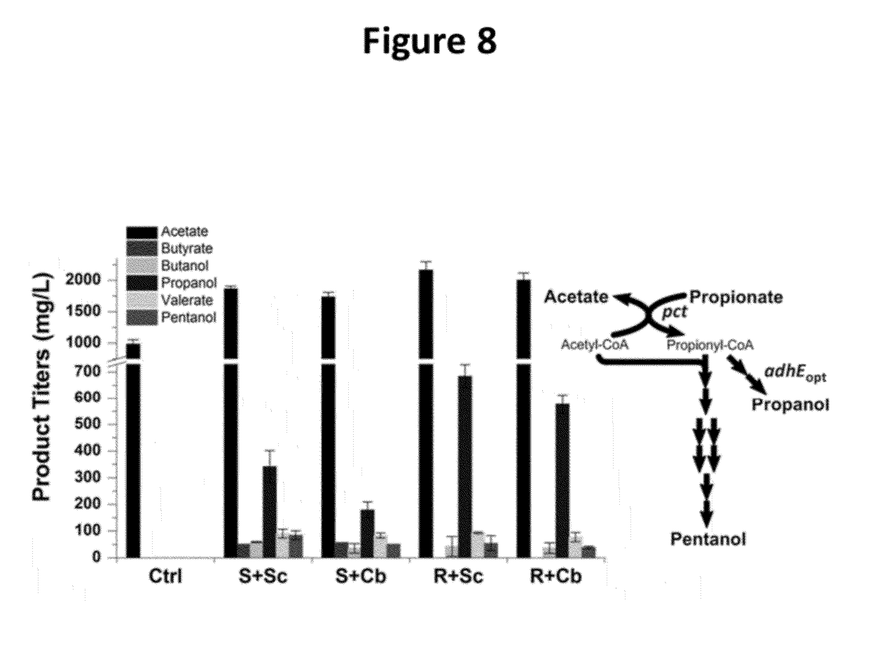

The pentanol pathway was employed for pentanol synthesis from glucose and propionate. Here, Pct was expressed to activate propionate to propionyl-CoA, and each of the Fdh1 enzymes was utilized to increase NADH availability. In addition, cell cultures were performed under the condition at a headspace to culture volume ratio of 4 because of the previous observation that such ratio achieved highest butanol specific titers. In general, a boosted acetate production was observed compared to the control containing empty plasmids (FIG. 8), most likely due to the activation of propionate to propionyl-CoA with concomitant production of acetate from acetyl-CoA. Also, the propionyl-CoA seemed to be converted to propanol by the AdhE enzyme as propanol was produced with titers up to 350 mg/L and 700 mg/L in the S- and R-constructs, respectively, indicating a promiscuity of the AdhE enzyme. More importantly, all recombinant strains yielded pentanol from glucose and propionate with titers up to 85 mg/L (FIG. 8).

### Example 7

### Pentanol Synthesis Solely from Glucose or Glycerol

Recombinant strains containing 4 compatible plasmids carrying all pentanol pathway genes were constructed and the resulting strains were tested for pentanol production solely from glucose or glycerol.

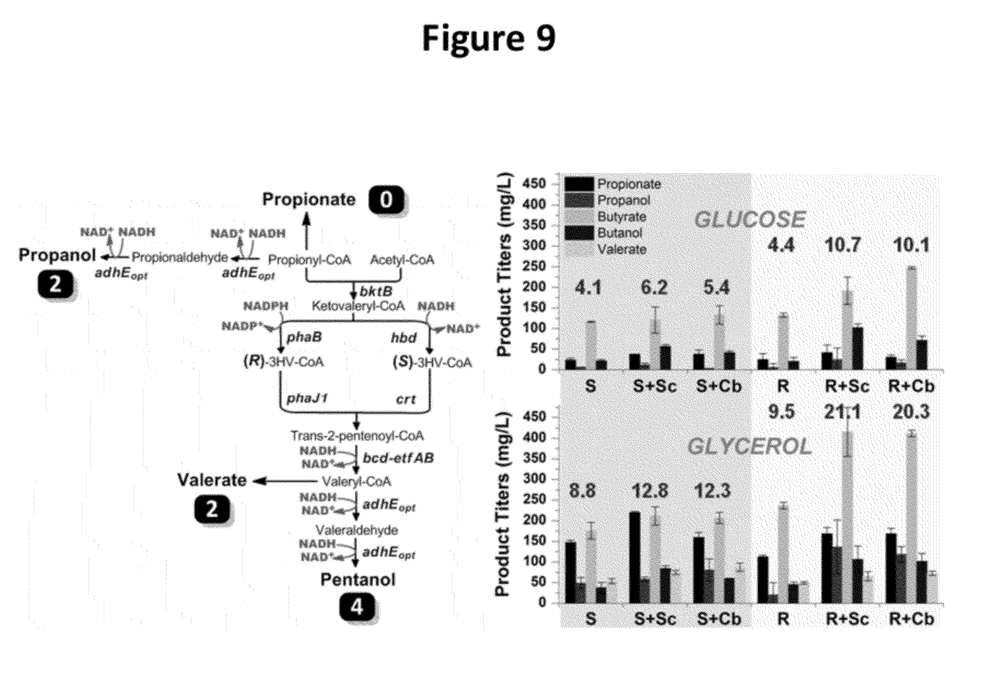

When glucose was used as the carbon source, only butyrate and butanol along with trace amount of propionate and propanol were produced. Neither valerate nor pentanol was synthesized (FIG. 9). By comparison, when glycerol was used as the sole carbon source, titers of propionate, propanol, butyrate, and butanol were significantly increased compared to glucose as the carbon source. Particularly, the boosted production of propionate and propanol suggest a sufficient supply of propionyl-CoA from glycerol. Direct valerate synthesis from glycerol was achieved. However, pentanol was not produced in either case.

The total NADH used for product formation was then calculated in a way similar to what was done previously, by summing up the products of multiplying the relative redox value by the product titer. Again, the over-expression of fdh1 was shown to enhance production of more reduced products. In addition, the values of total NADH used for product formation in glycerol cultures were much larger than those in glucose cultures, consistent with the fact that glycerol is more reduced than glucose.

### Example 8

### Materials and Methods for Examples 1-7

### Plasmids

Codon-optimized genes, including adhE from C. acetobutylicum ATCC 824, fdh1 from S. cerevisiae, and fdh1 from C. boidinii, were purchased from Genscript (Piscataway, N.J.). Genes derived from C. acetobutylicum ATCC 824 (hbd, crt, bcd, etfAB, adhE, and ptb-buk), R. eutropha H16 (bktB and phaB), P. aeruginosa (phaJ1), M. elsdenii (pct), C. glutamicum ATCC 13032 (ilvA), and E. coli ATCC 21277 (thrA^{G1297A}BC operon) were obtained by polymerase chain reaction (PCR) using genomic DNA (gDNA) templates. All gDNAs were prepared using the Wizard Genomic DNA Purification Kit (Promega, Madison, Wis.). Custom oligonucleotides (primers) were purchased for all PCR amplifications (Sigma-Genosys, St. Louis, Mo.). In the examples provided herein, PHUSION® High Fidelity DNA polymerase (Finnzymes, Espoo, Finland) was used for DNA amplification. Restriction enzymes and T4 DNA ligase were purchased from New England Biolabs (Ipswich, Mass.). Recombinant DNA techniques were performed according to standard procedures (Sambrook & Russell, 2001).

Compatible vectors pETDuet-1, pCDFDuet-1, pACYCDuet-1, and pCOLADuet-1 (Novagen, Darmstadt, Germany) were used to provide individual expression of each gene under a T7lac promoter and a ribosome binding site (RBS). These PCR products were digested with restriction enzymes corresponding to the restriction site incorporated into them by their respective primers and ligated directly into similarly digested Duet vectors. Ligation reactions using pETDuet-1, pACYCDuet-1, or pCOLADuet-1 as the vector were transformed into E. coli DH10B, while ligations using pCDFDuet-1 were transformed into E. coli ElectroTen-Blue. All constructs were confirmed to be correct by restriction enzyme digestion and nucleotide sequencing. Once all plasmids were constructed, they were co-transformed as appropriate into E. coli BL21Star(DE3) or Pal(DE3) to create production strains.

### Strains

E. coli DH10B (Invitrogen, Carlsbad, Calif.) and ElectroTen-Blue (Stratagene, La Jolla, Calif.) were used for transformation of cloning reactions and propagation of all plasmids. E. coli MG1655(Δpta ΔadhE ΔldhA) was kindly donated by Professor Gregory Stephanopoulos of the Department of Chemical Engineering at the Massachusetts Institute of Technology, USA. E. coli Pal(DE3) was then constructed from E. coli MG1655(Δpta ΔadhE ΔldhA) using a λDE3 Lysogenization Kit (Novagen, Darmstadt, Germany) to allow the expression of genes under the T7lac promoter (Fischer et al. Appl Microbiol Biotechnol 88, 265-275 (2010)). E. coli BL21Star(DE3) (Invitrogen, Carlsbad, Calif.) was used as the host strain for substrate feeding experiments, including pentanol synthesis from valerate (strains BL1-BL4, Table 2) or trans-2-pentenoate (strains BL5-BL7, Table 2) while Pal(DE3) was the production host strain employed for the rest of experiments, including trans-2-pentenoate synthesis from glucose and propionate (strains Pal1-Pal4, Table 2), butanol synthesis from glucose (strains Pal5 and Pal6, Table 2), pentanol synthesis from glucose and propionate (strains Pal7-Pal11, Table 2), and pentanol synthesis solely from glucose or glycerol (strains Pal12-Pal17, Table 2).

TABLE 2
E. coli strains and plasmids.

| Name | Relevant Genotype | Reference |
|---|---|---|
| Strains | | |
| DH10B | F⁻ mcrA Δ(mrr-hsdRMS-mcrBC) φ80lacZΔM15 ΔlacX74 recA1 endA1 | Invitrogen |
| | araD139Δ(ara, leu)7697 galU galK λ⁻ rpsL nupG | |
| ElectroTen-Blue | Δ(mcrA)183 Δ(mcrCB-hsdSMR-mrr)173 endA1 supE44 thi-1 recA1 | Stratagene |
| | gyrA96 relA1 lac Kan^{r} [F′ proAB lacI^{q}ZΔM15 Tn10 (Tet^{r})] | |
| BL21Star(DE3) | F⁻ ompT hsdS_{B} (r_{B}⁻ m_{B}⁻) gal dcm rne131 (DE3) | Invitrogen |
| BL1 | pCDF/ptb-buk + pACYC/adhE | This study |
| BL2 | pCDF/ptb-buk + pACYC/adhEₒₚₜ | This study |
| BL3 | pCDF/pct + pACYC/adhE | This study |
| BL4 | pCDF/pct + pACYC/adhEₒₚₜ | This study |
| BL5 | pET/bcd-etfAB/adhEₒₚₜ + pCDF/ptb-buk | This study |
| BL6 | pET/bcd-etfAB/adhEₒₚₜ + pCDF/ptb-buk/fdh1_{Sc} | This study |
| BL7 | pET/bcd-etfAB/adhEₒₚₜ + pCDF/ptb-buk/fdh1_{Cb} | This study |
| MG1655 | F⁻ λ⁻ ilvG- rfb-50 rph-1 | ATCC 700926 |
| Pal(DE3) | MG1655(DE3 Δpta ΔldhA ΔadhE) | (Fischer et al., 2010) |
| Pal1 | pET/bktB/hbd + pCDF/ptb-buk/crt | This study |
| Pal2 | pET/bktB/hbd + pCDF/ptb-buk | This study |
| Pal3 | pET/bktB/phaB + pCDF/ptb-buk/phaJ1 | This study |
| Pal4 | pET/bktB/phaB + pCDF/ptb-buk | This study |
| Pal5 | pET/bcd-etfAB/bktB + pCDF/crt/hbd + pACYC/adhEₒₚₜ | This study |
| Pal6 | pET/bcd-etfAB/bktB + pCDF/phaJ1/phaB + pACYC/adhEₒₚₜ | This study |
| Pal7 | pETDuet-1 + pCDFDuet-1 + pCOLADuet-1 | This study |
| Pal8 | pET/bcd-etfAB/bktB/pct + pCDF/crt/hbd + pACYC/fdh1_{Sc}/adhEₒₚₜ | This study |
| Pal9 | pET/bcd-etfAB/bktB/pct + pCDF/crt/hbd + pACYC/fdh1_{Cb}/adhEₒₚₜ | This study |
| Pal10 | pET/bcd-etfAB/bktB/pct + pCDF/phaJ1/phaB + pACYC/fdh1_{Sc}/adhEₒₚₜ | This study |
| Pal11 | pET/bcd-etfAB/bktB/pct + pCDF/phaJ1/phaB + pACYC/fdh1_{Cb}/adhEₒₚₜ | This study |
| Pal12 | pET/bcd-etfAB/bktB + pCDF/crt/hbd + pACYC/adhEₒₚₜ + | This study |
| | pCOLA/thrA^{fr}BC/ilvA | |
| Pal13 | pET/bcd-etfAB/bktB + pCDF/crt/hbd + pACYC/fdh1_{Sc}/adhEₒₚₜ + | This study |
| | pCOLA/thrA^{fr}BC/ilvA | |
| Pal14 | pET/bcd-etfAB/bktB + pCDF/crt/hbd + pACYC/fdh1_{Cb}/adhEₒₚₜ + | This study |
| | pCOLA/thrA^{fr}BC/ilvA | |
| Pal15 | pET/bcd-etfAB/bktB + pCDF/phaJ1/phaB + pACYC/adhEₒₚₜ + | This study |
| | pCOLA/thrA^{fr}BC/ilvA | |
| Pal16 | pET/bcd-etfAB/bktB + pCDF/phaJ1/phaB + pACYC/fdh1_{Sc}/adhEₒₚₜ + | This study |
| | pCOLA/thrA^{fr}BC/ilvA | |
| Pal17 | pET/bcd-etfAB/bktB + pCDF/phaJ1/phaB + pACYC/fdh1_{Cb}/adhEₒₚₜ + | This study |
| | pCOLA/thrA^{fr}BC/ilvA | |
| Plasmids | | |
| pETDuet-1 | ColE1(pBR322) ori, lacI, T7lac, Amp^{R} | Novagen |
| pCDFDuet-1 | CloDF13 ori, lacI, T7lac, Strep^{R} | Novagen |
| pACYCDuet-1 | P15A ori, lacI, T7lac, Cm^{R} | Novagen |
| pCOLADuet-1 | COLA ori, lacI, T7lac, Kan^{R} | Novagen |
| pET/bcd-etfAB/bktB | pETDuet-1 harboring bcd-etfAB operon from C. acetobutylicum ATCC | This study |
| | 824, and bktB from R. eutropha H16 | |
| pET/bcd-etfAB/bktB/pct | pETDuet-1 harboring bcd-etfAB operon from C. acetobutylicum ATCC | This study |
| | 824, bktB from R. eutropha H16, and pct from M. elsdenii | |
| pET/bcd-etfAB/adhEₒₚₜ | pETDuet-1 harboring bcd-etfAB operon and codon-optimized adhEₒₚₜ | This study |
| | from C. acetobutylicum ATCC 824 | |
| pET/bktB/hbd | pETDuet-1 harboring bktB from R. eutropha H16, and hbd from C. acetobutylicum | (Tseng et al., 2009*) |
| | ATCC 824 | |
| pET/bktB/phaB | pETDuet-1 harboring bktB and phaB from R. eutropha H16 | (Tseng et al., 2009*) |
| pCDF/crt/hbd | pCDFDuet-1 harboring crt and hbd from C. acetobutylicum ATCC 824 | This study |
| pCDF/phaJ1/phaB | pCDFDuet-1 harboring phaJ1 from P. aeruginosa, and phaB from R. eutropha | This study |
| | H16 | |
| pCDF/ptb-buk | pCDFDuet-1 harboring ptb-buk operon from C. acetobutylicum ATCC | (Tseng et al., 2009*) |
| | 824 | |
| pCDF/ptb-buk/fdh1_{Sc} | pCDFDuet-1 harboring ptb-buk operon from C. acetobutylicum ATCC | This study |
| | 824, and codon-optimized fdh1 from S. cerevisiae | |
| pCDF/ptb-buk/fdh1_{Cb} | pCDFDuet-1 harboring ptb-buk operon from C. acetobutylicum ATCC | This study |
| | 824, and codon-optimized fdh1 from C. boidinii | |
| pCDF/ptb-buk/crt | pCDFDuet-1 harboring ptb-buk operon and crt from C. acetobutylicum | This study |
| | ATCC 824 | |
| pCDF/ptb-buk/phaJ1 | pCDFDuet-1 harboring ptb-buk operon from C. acetobutylicum ATCC | This study |
| | 824, and phaJ1 from P. aeruginosa | |
| pCDF/pct | pCDFDuet-1 harboring pct from M. elsdenii | This study |
| pACYC/adhE | pACYCDuet-1 harboring adhE from C. acetobutylicum ATCC 824 | This study |
| pACYC/adhEₒₚₜ | pACYCDuet-1 harboring codon-optimized adhEₒₚₜ from C. acetobutylicum | This study |
| | ATCC 824 | |
| pACYC/fdh1_{Sc}/adhEₒₚₜ | pACYCDuet-1 harboring codon-optimized fdh1 from S. cerevisiae, and | This study |
| | codon-optimized adhEₒₚₜ from C. acetobutylicum ATCC 824 | |
| pACYC/fdh1_{Cb}/adhEₒₚₜ | pACYCDuet-1 harboring codon-optimized fdh1 from C. boidinii, and | This study |
| | codon-optimized adhEₒₚₜ from C. acetobutylicum ATCC 824 | |
| pCOLA/thrA^{fr}BC/ilvA | pCOLADuet-1 harboring thrA^{G1297A}BC operon from E. coli ATCC 21277, | (Tseng et al., 2010**) |
| | and ilvA from C. glutamicum ATCC 13032 | |

*Tseng et al. Appl. Environ. Microbiol. 75(10), 3137-3145 (2009)
**Tseng et al. Microb Cell Fact 9, 96. (2010)

### Culture Conditions

For trans-2-pentenoate synthesis from glucose and propionate, seed cultures of the recombinant strains (strains Pal1-Pal4) were grown in TB medium at 30° C. overnight on a rotary shaker at 250 rpm, and were used to inoculate, at an inoculation volume of 10%, 50 mL TB medium in 250 ml flasks for aerobic growth, and 13 ml TB medium in 15 mL glass tubes (Bellco Glass, Inc.) with a butyl rubber septum stopper for microaerobic or anaerobic growth. The septum was pierced with a 26-gauge syringe needle to achieve microaerobic conditions. All cell cultures were supplemented with 10 g/L glucose. Cultures were induced with 0.5 mM IPTG at 2 h post-inoculation and incubated for another 24 h.

For the substrate feeding experiments, seed cultures of the recombinant strains (strains BL1-BL7, Table 2) were grown in TB medium at 30° C. overnight on a rotary shaker at 250 rpm, and were used to inoculate 45 mL TB medium supplemented with 10 g/L glucose at an inoculation volume of 10% in 50 mL glass culture tubes. Cultures were induced with 0.5 mM IPTG at 2 h post-inoculation and incubated for another 72 h. One of the substrates, including neutralized valerate (10 mM or 20 mM) and trans-2-pentenoate (20 mM) was supplemented at the same time of induction to provide the precursor needed for pentanol synthesis.

For pentanol synthesis, seed cultures of the recombinant E. coli strains (strains BL7-BL17, Table 2) were grown in TB medium at 30° C. overnight, and were used to inoculate 3 mL TB medium supplemented with 10 g/L glucose or 10 g/L glycerol at an inoculation volume of 10% in 15 mL Falcon tubes. Cultures were induced with 0.5 mM IPTG at 2 h post-inoculation and incubated for another 96 h. For strains BL12-BL17 (Table 2), 20 mM neutralized propionate was supplemented at the same time of induction.

For profiling experiments with various headspace to culture volume ratios, the seed cultures of the recombinant strains (strains Pal5 and Pal6, Table 2) were used to inoculate, at an inoculation volume of 10%, 10 mL, 5 mL, 3 mL of TB medium in 15 mL Falcon tubes, 25 mL and 10 mL of TB medium in 250 mL flasks with caps screwed on, and 25 mL of TB medium in 250 mL flask with loose caps. All cultures were supplemented with 10 g/L glucose and then incubated at 30° C. on a rotary shaker. Cultures were induced with 0.5 mM IPTG at 2 h post-inoculation and incubated for another 48 h.

In all cases, culture medium was supplemented with 50 mg/L ampicillin, 50 mg/L streptomycin, 34 mg/L chloramphenicol, and 25 mg/L kanamycin as required. 1 mL of culture was withdrawn at the end of the incubation period for HPLC analysis. In general, experiments were performed in triplicates, and data are presented as the averages and standard deviations of the results.

### Metabolite Analysis

Culture samples were pelleted by centrifugation and aqueous supernatant collected for HPLC analysis using an Agilent 1200 series instrument with a refractive index detector (RID) and a diode array detector (DAD) at 210 nm. Analytes were separated using an Aminex HPX-87H anion-exchange column (Bio-Rad Laboratories, Hercules, Calif.) and a 5 mM H₂SO₄ mobile phase. Glucose, glycerol, acetate, 3-hydroxybutyrate, 3-hydroxyvalerate, crotonate, trans-2-pentenoate, butyrate, valerate, butanol, pentenol, and pentanol were quantified using commercial standards on a 1200 series by linear extrapolation from calibration of external standards.

### Example 9

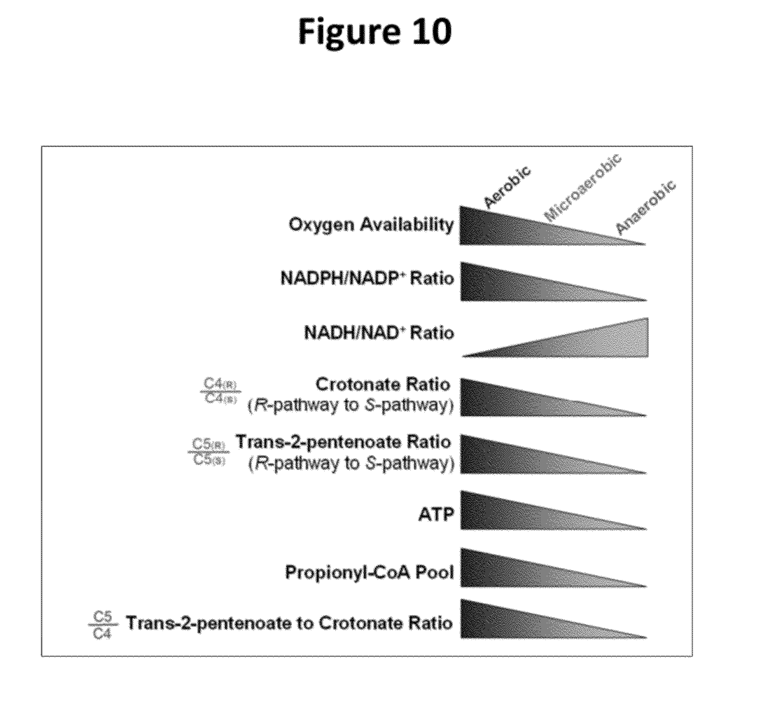

The results of trans-2-pentenoate synthesis from glucose and glycerol under aerobic, microaerobic, and anaerobic culture conditions suggest that there may exist a correlation between oxygen availability and R- and S-pathway activities, to explain which a hypothesis was proposed (FIG. 10).

As generally known, NADPH/NADP⁺ ratio is positively correlated with DOT (dissolved oxygen tension) while NADH/NAD⁺ ratio is inversely correlated with DOT, so the NADPH-dependent R-pathway was expected to outperforms the NADH-dependent S-pathway under aerobic conditions, resulting in larger crotonate ratios (R-pathway to S-pathway) and trans-2-pentenoate ratios (R-pathway to S-pathway) (FIG. 3). When it comes to anaerobic conditions, the trend of cofactor ratios reverses, the difference in performance between the two pathways should become smaller, which is consistent with what was observed. In addition, the activation of propionate was expected to be affected by DOT because its activation by Ptb-Buk requires ATP. The amount of ATP is positively correlated with DOT. Thus, a larger propionyl-CoA pool could be expected under the aerobic conditions, thus favoring the condensation reaction of acetyl-CoA with propionyl-CoA, and consequently resulting in larger trans-2-pentenoate to crotonate ratios (FIG. 3). Such observation provided insight into optimization of culture conditions, which motivated the profiling of the various headspace to culture volume ratios for production of butanol or pentanol (FIG. 7). In addition, this result and the other observation on undetectable pentanol production from the feeding of trans-2-pentenoate led to the hypothesis that pentanol biosynthetic pathway activity may be affected or limited by the intracellular NADH availability. To explore this hypothesis, the effect of overexpression of yeast fdh1 genes on pentanol synthesis from trans-2-pentenoate (FIG. 5) was investigated, and findings demonstrated pentanol production. The top and bottom pentanol pathway was assembled next and was introduced it to E. coli. The resulting recombinant E. coli synthesized pentanol from glucose and propionate, demonstrating a functional and feasible pentanol biosynthetic pathway in E. coli (FIG. 8).

Undetectable pentanol was observed in the experiment of direct production of pentanol solely from glucose or glycerol, most likely due to limited pathway activity as a result of limited availability of NADH. This is of particular concern with the pentanol biosynthetic pathway that combines an energetically expensive threonine biosynthesis pathway with the Clostridial butanol pathway, which has intensive requirement for reducing equivalent, consuming 4 moles NADH per mole of butanol produced. The intensive requirement for NADH and other reducing equivalents in pentanol synthesis was expected to create a redox imbalance in E. coli, thus making the pentanol biosynthetic pathway unpreferable. To improve the pathway performance, the redox imbalance was addressed, and one solution was to engineer E. coli to further boost the NADH availability.

Under anaerobic growth conditions, one mole of glucose yields only two moles of NADH due to an inactive pyruvate dehydrogenase complex (PDHc) enzyme. Therefore, a mutant was constructed that has an active PDHc under anaerobic conditions to boost NADH yield up to 4 moles of NADH per mole of glucose. Furthermore, the pentanol biosynthesis uses acetyl-CoA as one of precursor substrates that could possibly be limited in wild-type E. coli under anaerobic conditions. Therefore, an anaerobically active PDHc was also expected to enlarge acetyl-CoA pool, thereby enhancing the pentanol biosynthesis. An E. coli mutant SE2378 has been isolated with a mutant PDHc that functions under anaerobic conditions (Kim et al. J Bacteriol 190, 3851-3858 (2008)). In such case, four NADH molecules were re-oxidized by reduction of the two acetyl-CoA molecules to produce two ethanol molecules at a yield of 82% from glucose. SE2378 demonstrated poor transformation efficiency as well as other undesired phenotypes resulting from random mutagenesis that could prevent from further strain development. Construction of a genetically-defined E. coli strain with a similar phenotype to mutant SE2378 is an option to enriched NADH and acetyl-CoA pools.

### Example 10

### Use of Ter Gene for Pentanol Synthesis

Based on the work of Tucci and Martin describing the Trans-2-enoyl-CoA reductase (Ter) (Tucci and Martin, 2007), the Ter enzyme from T. denticola works on C4 (crotonyl-CoA) substrates but not C6 (trans-hexenoyl-CoA) substrates with a specific activity of 43.4±4.8 U/mg (μmo/mg/min) using crotonyl-CoA as the substrate. On the other hand, the Ter from E. gracilis works on both C4 and C6 substrates, but its specific activity on crotonyl-CoA (3.9 U/mg) is 10-fold lower than Ter from T. denticola. In addition, the Ter enzyme from T. denticola showed NADH-dependent activity while the Ter enzyme from E. gracilis accepts both NADH and NADPH. More importantly, the Ter enzyme from T. denticola and the Ter enzyme from E. gracilis both have undetectable oxidizing activity in the reverse reaction when butyryl-CoA was utilized as the substrate while Bcd from C. acetobutylicum has been shown to have the oxidizing activity, which could result in a kinetic bottleneck for the pentanol biosynthesis. Considering the distinct characteristics of both Ter enzymes with some superior features than the Bcd enzyme, cells were engineered to express one of each Ter gene, in place of Bcd, to catalyze the reaction of trans-2-pentenoyl-CoA to valeryl-CoA.

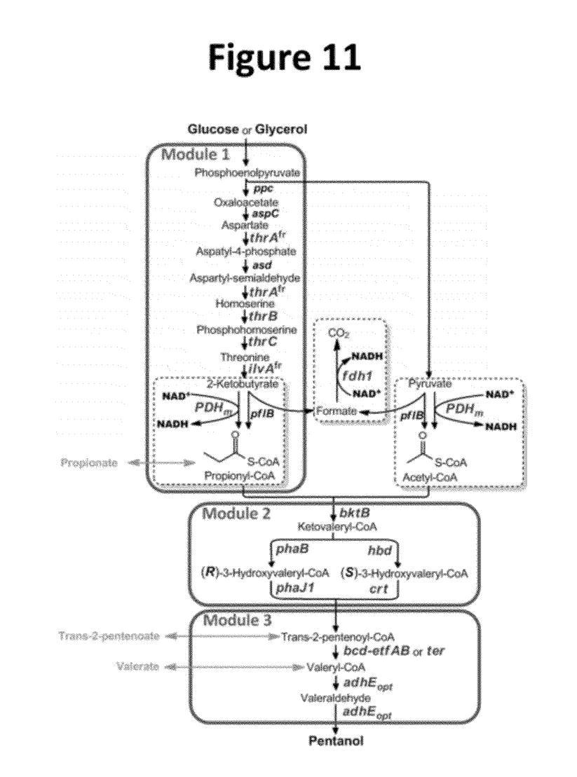

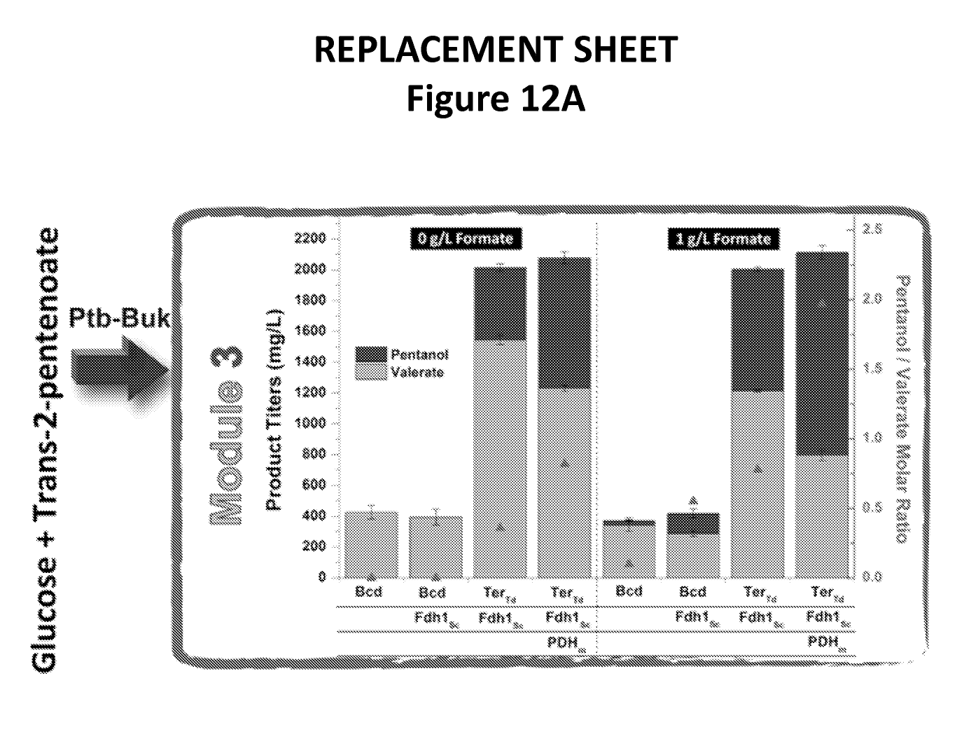

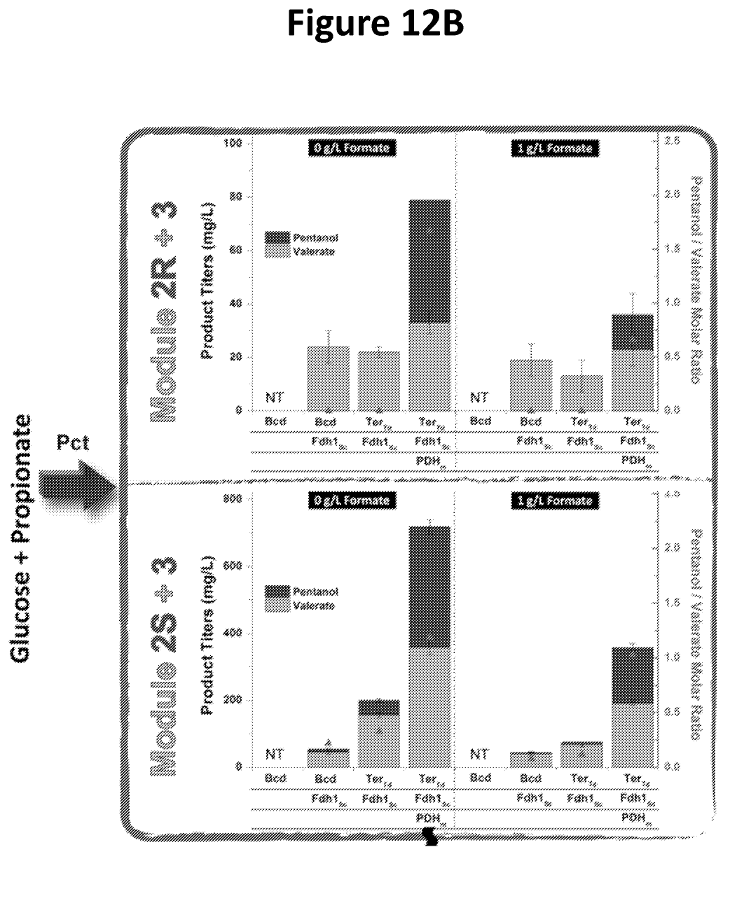

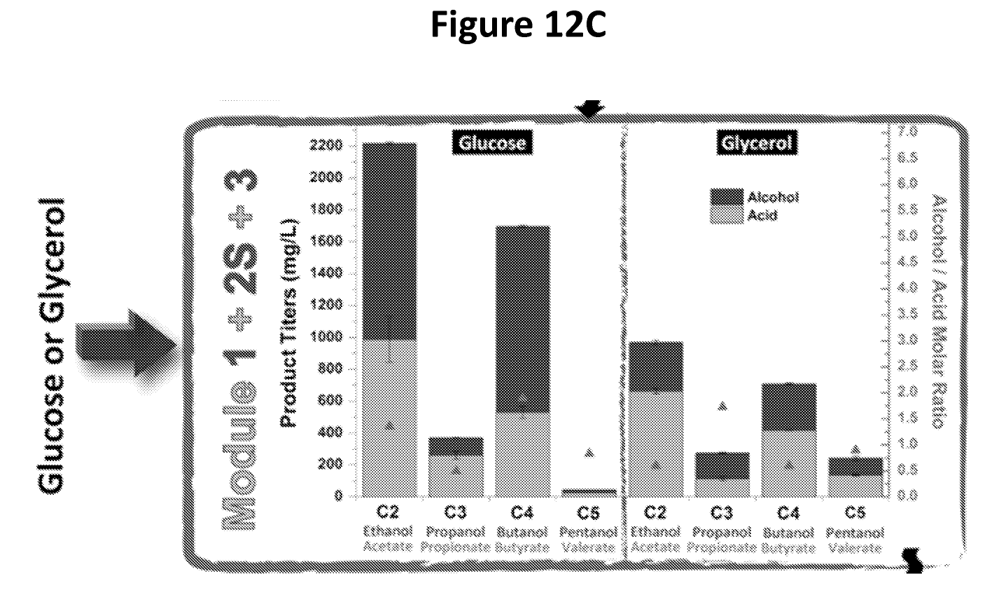

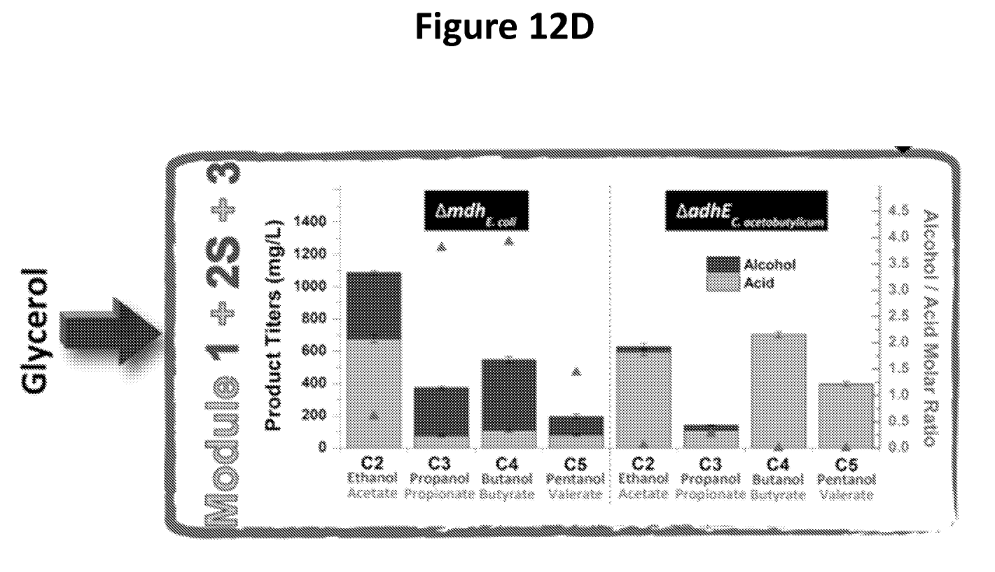

The pentanol biosynthetic pathway can be defined as containing three shorter pathways or “modules”, each of which was validated separately and then assembled together (FIG. 11). In Module 3, trans-2-pentenoate was supplemented exogenously and activated to trans-2-pentenoyl-CoA by the CoA-activator Ptb-Buk, followed by sequential conversion to pentanol, catalyzed by Bcd and AdhEₒₚₜ. Module 3 produced valerate but not pentanol (FIG. 12A, and top left plot in FIG. 5), most likely due to insufficient NADH supply. Pentanol synthesis from valeryl-CoA requires two molecules of NADH while valerate synthesis from valeryl-CoA, a non-redox reaction, does not require NADH. Additionally, production of valerate through Ptb-Buk yields one ATP. Thus, production of valerate would be energetically more favorable than pentanol. If this hypothesis is correct, increased NADH availability should facilitate pentanol production.

To increase the availability of NADH, three experiments were conducted. First, yeast NAD⁺-dependent formate dehydrogenase, encoded by the fdh1 gene (Berrios-Rivera et al., 2002), was overexpressed. Second, the Bcd enzyme was replaced with one of the Ter enzymes, which directly uses NADH as the electron donor (Bond-Watts et al. Nat Chem Biol 7, 222-7 (2011); Shen et al. Appl. Environ. Microbiol. 77, 2905-2915 (2011)). The Ter enzyme from T. denticola accepts C4 (crotonyl-CoA) but not C6 (trans-hexenoyl-CoA) substrates (Tucci and Martin, 2007). By comparison, Ter from E. gracilis accepts both C4 and C6 substrates, but its specific activity on crotonyl-CoA is 10-fold lower than Ter from T. denticola. Neither enzyme had been profiled against C5 substrates. Third, the anaerobically active PDH mutant lpd101-E354K (PDHₘ) (E. coli mutant SE2378 strain discussed above; a gift from K. T. Shanmugam, Department of Microbiology and Cell Science, University of Florida) (Kim et al., 2008) was overexpressed, which was expected to boost the NADH yield on glucose from two moles to four moles of NADH per mole of glucose.

Codon-optimized fdh1 genes from Saccharomyces cerevisiae and Candida boidinii were initially tested. In formate-supplemented cultures, the overexpression of either Fdh1_{Sc} or Fdh1_{Cb} resulted in the synthesis of more reduced products, including pentenol and pentanol (bottom three plots in FIG. 5). The two codon-optimized ter genes from T. denticola (Ter_{Td}) and E. gracilis (Ter_{Eg}) were then compared, and Ter_{Td} was found to enhance pentanol synthesis much more than Ter_{Eg}. Overexpression of Fdh1_{Sc}, Ter_{Td} and PDHₘ resulted in pentanol titers up to 1317 mg/L from 2 g/L trans-2-pentenoate in the presence of 1 g/L formate (FIG. 12A). Overall, the NADH deficiency problem was addressed and there was a drastic improvement in pentanol production from an undetectable amount to more than 1 g/L.

After validating all three modules, they were linked together to produce the C5 alcohol from a single carbon source. Modules 2 and 3 were first assembled, resulting in up to 46 mg/L and 358 mg/L of pentanol, respectively, through the phaB-phaJ1 (module 2R+3) or hbd-crt routes (module 2S+3) (FIG. 12B). These titers are between those obtained when only module 2 was employed to produce trans-2-pentenoate and when only module 3 was used to produce pentanol. These results validate previous reports that emphasize the importance of considering the complete pathway for optimization of productivity (Ajikumar, P. K., et al. Science 330, 70-4 (2010)) while also suggesting that the full synthetic capacity of module 3 is being underutilized, perhaps due to increased cofactor demands when modules 2 and 3 are combined or because of limited flux through Crt/PhaJ1. Because the hbd-crt pathway produced a higher concentration of pentanol than that produced by the phaB-phaJ1 pathway, the former was chosen for further study. Recombinant strains containing all three modules (module 1+2S+3) synthesized 19 mg/L of pentanol from glucose and 109 mg/L of pentanol from glycerol (FIG. 12C). The lower titers from a sole carbon source indicate a need to increase the precursor supply. The results demonstrated a functional and feasible pentanol biosynthetic pathway from a sole carbon source in E. coli.

To further improve alcohol synthesis, the mdh gene, encoding malate dehydrogenase that converts oxaloacetate to malate using NADH as a cofactor, was deleted. The mdh deletion led to product redistribution towards alcohols with increased alcohol/acid molar ratios (FIG. 12D). The product distribution was also shifted in the opposite direction by removing the adhE gene in the pentanol biosynthetic pathway, resulting in near abolishment of alcohol synthesis (FIG. 12D).

### Equivalents and Scope

Those skilled in the art will recognize or be able to ascertain using no more than routine experimentation many equivalents to the specific embodiments described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, or up to 1% of a given value. Alternatively, the term can mean within an order of magnitude, for example, within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the methods of the invention can be excluded from any one or more claims, for any reason, whether or not related to the existence of prior art.

Each of the foregoing patents, patent applications and references is hereby incorporated by reference, particularly for the teaching referenced herein.

We claim:

1. A cell that recombinantly expresses one or more genes of the butanol biosynthetic pathway and a gene encoding a thiolase that condenses one acetyl-CoA with one propionyl-CoA to form a ketone.

2. The cell of claim 1 that recombinantly expresses:
(a) a gene encoding a thiolase that condenses one acetyl-CoA with one propionyl-CoA to form a ketone;
(b) genes encoding a reductase, a hydratase, and a dehydrogenase that convert the ketone to valeryl-CoA; and
(c) a gene encoding a bi-functional aldehyde/alcohol dehydrogenase.

3. The cell of claim 2 that recombinantly expresses:
(a) a gene encoding an acetoacetyl-CoA thiolase that condenses one acetyl-CoA with one propionyl-CoA to form 3-ketovaleryl-CoA;
(b) genes encoding a 3-hydroxybutyryl-CoA reductase, an enoyl-CoA hydratase, and a butyryl-CoA dehydrogenase; and
(c) a gene encoding a bi-functional aldehyde/alcohol dehydrogenase.

4. The cell of claim 1 that recombinantly expresses:
(a) a bktB gene;
(b) a hbd gene and a crt gene, or
a phaB gene and a phaJ1 gene;
(c) a bcd-etfAB gene pair; and
(d) an adhE gene.

5. The cell of claim 4, wherein the cell does not express an mdh gene.

6. (canceled)

7. The cell of claim 1, wherein the cell recombinantly expresses a thrA^{fr} gene, a thrB gene, a thrC gene, and a ilvA^{fr} gene.

8. The cell of claim 7, wherein the cell endogenously or recombinantly expresses a gene encoding pyruvate dehydrogenase complex (PDHc) or a gene encoding pyruvate-formate lyase (PfIB).

9. The cell of claim 7, wherein the cell endogenously or recombinantly expresses a gene encoding anaerobically active PDHc.

10. The cell of claim 1, wherein the cell recombinantly expresses a ptb-buk gene pair or a pct gene.

11. The cell of claim 1, wherein the cell is a bacterial cell, a fungal cell (including a yeast cell), a plant cell, an insect cell, or an animal cell.

12. The cell of claim 11, wherein the cell is a bacterial cell.

13-15. (canceled)

16. The cell of claim 4, wherein the hbd gene, crt gene, bcd-etfAB gene pair, and/or adhE gene(s) is a Clostridium acetobutylicum gene.

17. The cell of claim 4, wherein the bktB gene and/or phaB gene(s) is a Ralstonia eutropha gene.

18. (canceled)

19. The cell of claim 4, wherein the phaJ1 gene is a Pseudomonas aeruginosa gene.

20. The cell of claim 1, wherein the cell produces pentanol.

21. Cell culture medium or supernatant collected from culturing one or more cell(s) of claim 1.

22. A method, comprising culturing one or more cells of claim 1 in cell culture medium.

23-27. (canceled)

28. The method of claim 22, further comprising recovering pentanol from the cell or from the culture medium in which the cell is grown.

29. A method, comprising recombinantly expressing in a cell one or more genes of the butanol biosynthetic pathway and a thiolase that condenses one acetyl-CoA with one propionyl-CoA to form a ketone.

30-55. (canceled)

56. A cell that recombinantly expresses:
(a) a gene encoding an acetoacetyl-CoA thiolase that condenses one acetyl-CoA with one propionyl-CoA to form 3-ketovaleryl-CoA;
(b) genes encoding 3-hydroxybutyryl-CoA reductase, an enoyl-CoA hydratase, and a trans-enoyl-CoA reductase; and
(c) a gene encoding a bi-functional aldehyde/alcohol dehydrogenase.

57. The cell of claim 56 that recombinantly expresses:
(a) a bktB gene;
(b) a hbd gene and a crt gene, or
a phaB gene and a phaJ1 gene;
(c) a Ter gene; and
(d) an adhE gene.

58-108. (canceled)
